(19) United States
(12) Patent Application Publication
(10) Pub. No.: US 2011/0306639 A1
(43) Pub. Date: Dec. 15, 2011

(54) HYDROCARBYLIDENE NITROHYDROZINECARBOXIMIDAMIDES AND A METHOD FOR MAKING THE SAME, AS WELL AS THEIR USES AS AN INSECTICIDE

(76) Inventors: ZhaoHai Qin, Beijing (CN); Yongqiang Ma, Beijing (CN); Wangcang Su, Beijing (CN); Lei Wang, Beijing (CN); Zheng Zhang, Beijing (CN); Bengbin Zhao, Beijing (CN); Jiangsheng Fang, Beijing (CN)

(21) Appl. No.: 12/740,673

(22) PCT Filed: Nov. 25, 2008
(86) PCT No.: PCT/CN2008/001919

§ 371 Date: Sep. 2, 2011

(51) Int. Cl. A01N 43/40; A01P 7/04; C07D 405/12; A01N 47/44; C07C 281/16; C07D 213/53

(52) U.S. Cl. 514/336; 564/108; 546/332; 546/283.4; 514/610; 514/357

(57) ABSTRACT

The present invention discloses hydrocarbylidene nitrohydrozinecarboximidamides and the use thereof as well as a method for making the same. The structural general formula of the compounds are shown in formula I, wherein, R1 is C1-C10 saturated and/or unsaturated aliphatic hydrocarbonyl, benzyl, substituted benzyl, halogenated picolyl, halogenated thiazolyl methyl, tetrahydrofuryl methyl or oxazolyl methyl; R2 is hydrogen, C1-C5 saturated and/or unsaturated aliphatic hydrocarbonyl, phenyl, substituted phenyl, pyridyl or substituted pyridyl; R3 is hydrogen, C1-C10 saturated and/or unsaturated aliphatic hydrocarbonyl, furyl, phenyl, substituted phenyl, benzyl or substituted benzyl. The tests of insecticidal activity show that the hydrocarbylidene nitrohydrozinecarboximidamides shown by formula (I) have high preventive efficiency against insect pests of plants, such as aphid, plant hopper, cotton bollworm, asparagus caterpillar, and the like, and can be used as plant insecticides.

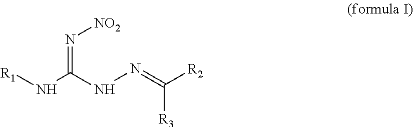

### FIELD OF THE INVENTION

The present invention relates to hydrocarbylidene nitrohydrozinecarboximidamides and a method for making the same, as well as their uses as an insecticide.

### BACKGROUND OF THE INVENTION

Nicotine is a natural alkaloid, which has been used as an insecticide as early as the nineteen century, and the target which acts on is the postsynaptic nicotinic acetylcholine receptor (nAchRs). However, the insecticidal activity of Nicotine is low, and it is highly toxic to human beings. With the Nicotine being as a leading compound, such insecticides have been fully developed and successive several generations of products have been commercially developed, since Bayer AG successfully developed the neonicotiniod insecticides, Imidacloprid, in 1980s. Neonicotine insecticides have high insecticidal activities and broad insecticidal spectrum, and are safe to mammalian and other environmental lives. The above insecticides have structural general formula as shown in formula A.

In contrast, the use of semicarbazone compounds as insecticides is developed a little late, however, the development of such insecticides has become a new hotspot since Dupont Corporation successfully developed indoxacarb in 1992. The action target of such insecticides is sodium ion channel, and they specially effect on nearly all of Lepidoptera pest, but they are safe to each of mammalian, avian and aquatic animal. Such insecticides have the structural general formula as shown in Formula B.

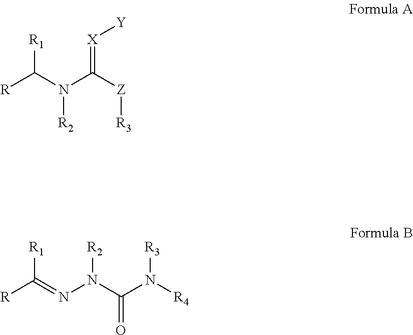

As to the biocompatibility, insecticides acting on a single target generally quickly result in resistance, and insecticides acting on multiple targets do slowly.

### SUMMARY OF THE INVENTION

The object of the present invention is to provide hydrocarbylidene nitrohydrozinecarboximidamides and a method for making the same.

The present invention provides hydrocarbylidene nitrohydrozinecarboximidamides, which has structural general formula as shown in formula I:

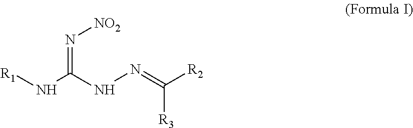

Wherein:

R1 is C1-C10 saturated and/or unsaturated aliphatic hydrocarbonyl, benzyl, substituted benzyl, halogenated picolyl, halogenated thiazolyl methyl, tetrahydrofuryl methyl or oxazolyl methyl, wherein the substituent of said substituted benzyl can be halogen (specifically F, Cl, Br, and I), amino, hydroxy, C1-C5 alkyl or C1-C5 alkoxyl, and the like;

R2 is hydrogen, C1-C5 saturated and/or unsaturated aliphatic hydrocarbonyl, phenyl, substituted phenyl, pyridyl or substituted pyridyl, wherein the substituent of said substituted phenyl can be halogen, hydroxy, amino, C1-C5 alkyl, C1-C5 alkoxyl, aryl (such as phenyl, pyridyl, imidazolyl, oxazolyl, and thiazyl), aryloxy (such as phenoxy, and pyridinyloxy) and the like, and the substituent of said substituted pyridyl can be halogen, C1-C5 alkyl, C1-C5 alkoxyl, aryloxy (such as phenoxy, and pyridinyloxy) and the like;

R3 is hydrogen, C1-C10 saturated and/or unsaturated aliphatic hydrocarbonyl, furyl, phenyl, substituted phenyl, benzyl or substituted benzyl, wherein the substituent of said substituted phenyl can be halogen, hydroxy, amino, substituted amino (such as methylamino, and dimethylamino), nitro, C1-C5 alkyl, C1-C5 alkoxyl, aryl (such as phenyl, pyridyl, imidazolyl, oxazolyl, thiazyl), aryloxy (such as phenoxy and pyridinyloxy) and the like, and the substituent of said substituted benzyl can be halogen, hydroxy, amino, C1-C5 alkyl, C1-C5 alkoxyl, aryl (such as phenyl, pyridyl, imidazolyl, oxazolyl, and thiazyl), aryloxy (such as phenoxy and pyridinyloxy), and the like;

Preferred R1 is C1-C10 unsaturated aliphatic hydrocarbonyl, halogenated picolyl, halogenated thiazolyl methyl or tetrahydrofuryl methyl; preferred R2 is hydrogen or C1-C5 saturated and/or unsaturated aliphatic hydrocarbonyl; and preferred R3 is substituted phenyl or C1-C10 saturated and/or unsaturated aliphatic hydrocarbonyl;

More preferred R1 is allyl, propargyl or chloro-picolyl; more preferred R2 is hydrogen; and more preferred R3 is substituted phenyl or C3-C7 saturated and/or unsaturated aliphatic hydrocarbonyl.

The saturated and/or unsaturated aliphatic hydrocarbonyl of the present invention can be linear or branched.

The present invention provides a method for making the hydrocarbylidene nitrohydrozinecarboximidamides shown in structural general formula I, including the following steps:

1) Reacting nitroguanidine with hydrazine hydrate to form N′-nitrohydrazinecarboximidamide shown by formula II;

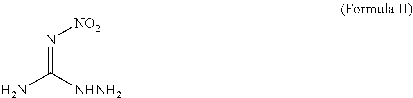

2) Reacting N′-nitrohydrazinecarboximidamide shown by formula II with carbonyl compounds shown by the structural general formula III under acid catalysis, to form hydrocarbylidene nitrohydrozinecarboximidamides shown by the structural general formula IV:

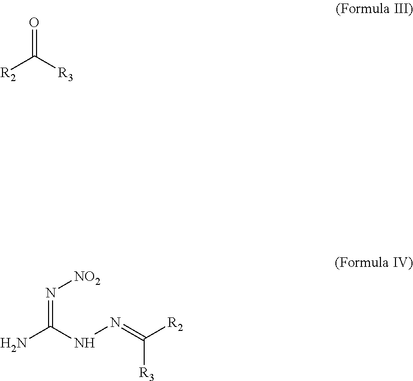

3) Reacting the compounds shown by the structural general formula IV with compounds shown by the structural general formula V (halohydrocarbons or sulfonic esters) under alkali catalysis, to form compounds shown by the structural general formula I;

R1—X (Formula V)

Wherein, R2 of formula II and formula III are hydrogen, C1-C5 saturated and/or unsaturated aliphatic hydrocarbonyl, phenyl, substituted phenyl, pyridyl or substituted pyridyl, wherein the substituent of said substituted phenyl can be halogen, hydroxy, amino, C1-C5 alkyl, C1-C5 alkoxyl, aryl (such as phenyl, pyridyl, imidazolyl, oxazolyl, and thiazyl), aryloxy (such as phenoxy, and pyridinyloxy) and the like, and the substituent of said substituted pyridyl can be halogen, C1-C5 alkyl, C1-C5 alkoxyl, aryloxy (such as phenoxy, and pyridinyloxy), and the like;

R3 is hydrogen, C1-C10 saturated and/or unsaturated aliphatic hydrocarbonyl, furyl, phenyl, substituted phenyl, benzyl or substituted benzyl, wherein the substituent of said substituted phenyl can be halogen, hydroxy, amino, substituted amino (such as methylamino, and dimethylamino), C1-C5 alkyl, C1-C5 alkoxyl, aryl (such as phenyl, pyridyl, imidazolyl, oxazolyl, and thiazyl), aryloxy (such as phenoxy, and pyridinyloxy) and the like, and the substituent of said substituted benzyl can be halogen, hydroxy, amino, C1-C5 alkyl, C1-C5 alkoxyl, aryl (such as phenyl, pyridyl, imidazolyl, oxazolyl, and thiazyl), aryloxy (such as phenoxy, and pyridinyloxy), and the like;

R1 of formula V is C1-C10 saturated and/or unsaturated aliphatic hydrocarbonyl, benzyl, substituted benzyl, halogenated picolyl, halogenated thiazolyl methyl, tetrahydrofuryl methyl or oxazolyl methyl, wherein the substituent of said substituted benzyl can be halogen, amino, hydroxy, C1-C5 alkyl or C1-C5 alkoxyl and the like, and X of formula V is Cl, Br, I, OTos (p-tosyloxy) or OTf (trifluoromethane sulfonyl);

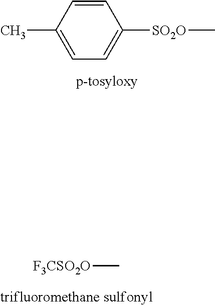

Preferred R1 is C1-C10 unsaturated aliphatic hydrocarbonyl, halogenated picolyl, halogenated thiazolyl methyl or tetrahydrofuryl methyl, preferred R2 is hydrogen or C1-C5 saturated and/or unsaturated aliphatic hydrocarbonyl; preferred R3 is substituted phenyl or C1-C10 saturated and/or unsaturated aliphatic hydrocarbonyl;

More preferred R1 is allyl, propargyl or chloro-picolyl, more preferred R2 is hydrogen, more preferred R3 is substituted phenyl or C3-C7 saturated and/or unsaturated aliphatic hydrocarbonyl.

The reaction in step 1) is conducted in a solvent, and said solvent can be water; and the reaction temperature of said reaction is 45-70° C. The molar ratio of nitroguanidine to hydrazine hydrate in step 1) is 1:1-1:1.5.

The reaction in step 2) is conducted in a solvent, and said solvent can be anhydrous ethanol or methanol; the reaction temperature of said reaction can be 50-80° C.; and the acids used in said reaction can be acetic acid or p-toluenesulfonic acid. The molar ratio of N′-nitrohydrazinecarboximidamide shown by formula II to carbonyl compounds shown by the structural general formula III in step 2) is 1:1-1:2.

The reaction in step 3) is conducted in a solvent, and said solvent can be DMF (dimethylformamide) or DME (dimethylacetamide); the reaction temperature of said reaction can be 0-50° C.; and the alkalies used in said reaction can be sodium hydride, sodium ethoxide, sodium methoxide or sodium amide. The molar ratio of the compounds shown by the structural general formula IV to the compounds shown by the structural general formula V in step 3) is (1:1.2)-(1:2.5).

Another object of the present invention is to provide the use of hydrocarbylidene nitrohydrozinecarboximidamides shown by the structural general formula I.

The present invention provides the use of hydrocarbylidene nitrohydrozinecarboximidamides shown by the structural general formula I, wherein the compounds shown by the structural general formula I or pharmaceutically acceptable salts thereof or pharmaceutical compositions containing any of them can be used to prepare plant insecticides.

A further object of the present invention is to provide a plant insecticidal drug or formulation.

The active ingredient of plant insecticidal drugs or formulations provided by the present invention is hydrocarbylidene nitrohydrozinecarboximidamides shown by the structural general formula I or pharmaceutically acceptable salts thereof.

The mass percent content of the active ingredient of said insecticidal drugs or formulations is 0.01%-99.99%.

Said insecticides can be processed into any acceptable dosage form as required. For example, the dosage form can be suspension, emulsion, aerosol, wettable powder, emulsifiable concentrate, and granule.

The preparation methods of dosage form are exemplified as follows:

The preparation of the suspension: the content of active ingredient in the conventional formulation is 5%-35%. With the water being media, active pharmaceutical ingredients, water dispersant, suspending agent and antifreeze agent and the like, are added into the sander and are ground, to prepare suspension.

The preparation of the wettable powder: according to the requirement of formulation, active pharmaceutical ingredients, a variety of surfactants and solid diluents, and the like, are fully mixed, and after ultrafinely grinding, the wettable powder product of predetermined content can be obtained. In order to prepare the wettable powder suitable for spraying, active pharmaceutical ingredients and comminuted solid powder such as clay, inorganic silicate, carbonate and wetting agent, adhesive and/or dispersant can also constitute a mixture.

The preparation of the emulsifiable concentrate: according to the requirement of formulation, the active ingredients are dissolved into organic solvents, and emulsifying agents and other adjuvants are added and processed to form the formulation. Solvents can be toluene, xylene, methanol, and the like, and a cosolvent if necessary; and the other adjuvants including stabilizer, permeate agent and erosion inhibitor, and the like.

The compounds shown by the structural general formula I provided by the present invention and the insecticidal drugs or formulations with said compounds being active ingredient can control and kill a broad range of pests, which includes sucking insects, biting insects and other plant pests, pests of grain storage, and sanitary pests causing health hazard, and the like.

The pests are exemplified as follows:

Homoptera pests include aphididae, aleyrodidae, delphacidae, psyllidae, jassidae and coccidae pests. Aphididae pests can specifically be aphis gossypii, bean aphid, myzus persicae, hyalopterus pruni, turnip aphid or cabbage aphid; delphacidae pests can specifically be nilaparvata lugens or rice planthopper; aleyrodidae pests can specifically be bemisia tabaci gennadius; jassidae pests can specifically be rice leafhopper (Nephotettix bipunctatus); and coccidae pests can specifically be arrowhead scale (Unaspis yanonensis).

Lepidoptera pests include noctuidae and plutellidae pests. Noctuidae pests can specifically be asparagus caterpillar, Spodoptera litura Fab or Helicoverpa armigera; plutellidae pests can specifically be Plutella xylostella.

The plant insecticidal drugs or formulations provided by the present invention can be used to prevent insect pests of plants. Especially, the plant insecticidal drugs or formulations have special efficiency against piercing-sucking type pests, scratching type mouthparts pests, such as various aphid, plant hopper, leafhopper, mealworm and Thrips palmi, and have high efficiency against Helicoverpa armigera and asparagus caterpillar.

A further object of the present invention is to provide a method for preventing insect pests of plants.

The method for preventing from insect pests of plants provided by the present invention is to apply the plant insecticidal drugs or formulations provided by the present invention to plant leaves and/or plant fruits and/or plant seeds, and the places where the plant leaves and/or plant fruits and/or plant seeds are growing or are expected to be grown. The active ingredient of the plant insecticidal drugs or formulations is administrated at the concentration of 1-600 mg/L, and preferably, at the concentration of 3-50 mg/L.

### THE BEST MODES OF CARRYING OUT THE INVENTION

Hydrocarbylidene nitrohydrozinecarboximidamides of the present invention can be synthesized by following steps:

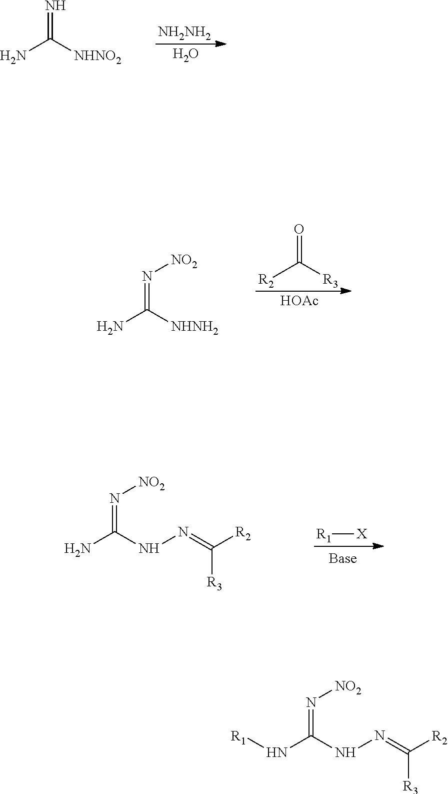

The present invention is further illustrated in combination with the following specific examples. It should be understood that these examples are used only to illustrate the present invention, but not to limit the scope of the present invention. In the following examples, experimental methods which do not indicate particular conditions usually are carried out under normal conditions or under the conditions proposed by the manufacturer. Unless otherwise specified, percentage and parts are calculated based on the mass.

In the following, using 2-isobutylidene-N′-nitrohydrozinecarboximidamide and 2-(2′-nitrophenyl methylene)-N-nitrohydrozinecarboximidamide as examples, the preparation method of hydrocarbylidene nitrohydrozinecarboximidamides provided by formula (I) of the present invention is illustrated.

### EXAMPLE 1

### The synthesis of 2-isobutylidene-N′-nitrohydrozinecarboximidamide (the compound 1 of formula I, wherein R1 is methyl, R2 is hydrogen, and R3 is isopropyl)

### (1) the synthesis of N′-nitrohydrozinecarboximidamide

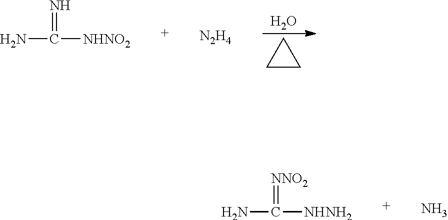

To 250 mL three-necked flask, 5.0 g (0.048 mol) nitroguanidine and 70 mL water were sequentially added. It was heated to 55° C. under magnetic stirring, and the aqueous solution of 85% by mass of hydrazine hydrates (wherein, the mass of the hydrazine hydrates added was 3.5 g (0.059 mol)) was slowly added dropwise through a drop funnel. The reaction was continued for 20 minutes, while the temperature of materials was kept between 55 and 60° C. When the materials turned into an orange clear liquid, it was cooled quickly with an ice water bath, and about 6 mL concentrated HCl (the mass percent is 37%) was slowly added dropwise to adjust pH as 5-6; the materials were continued to be cooled to 2-3° C. and lasted for 1 hour. The resulting product was filtered under reduced pressure, and washed with a little ice water, and air-dried in a fume hood. The resulting product was recrystallized with hot water, and 2.74 g light yellow powder (N′-nitro amino guanidine) was obtained in 48% yield, and the melting point of which is 191-192° C.

Structural characteristic data are provided as follows:

¹H NMR (DMSO-d₆, δppm): 4.69 (s, 2H, —NHNH₂), 7.56 (s, 1H, —NHNH₂), 8.27 (s, 1H, —NHNO₂), 9.33 (s, 1H, C═NH).

### (2) The synthesis of 2-isobutylidene-N′-nitrohydrozinecarboximidamide

To 250 mL three-necked flask, 24 g (0.2 mol) N′-nitrohydrozinecarboximidamide, 100 mL anhydrous ethanol, and 2.4 mL glacial acetic acid were sequentially added. It was heated to 65° C. under magnetic stirring, and 19.0 g (0.24 mol) isobutylaldehyde (the compounds in formula III, wherein R2 is hydrogen, and R3 is isopropyl) was slowly added dropwise through a drop funnel. After the addition was complete, the mixture was heated to reflux, and the reaction was refluxed for 3 hours. The temperature was lowered, and the solvent was removed under the reduced pressure. The resulting crude product was recrystallized with ethanol-petroleum ether (3:1 by volume) to obtain 20 g light yellow powder (2-isobutylidene-N′-nitrohydrozinecarboximidamide) in 52% yield, and the melting point of which is 76-78° C.

### (3) The synthesis of 2-isobutylidene-N-methyl-N′-nitrohydrozinecarboximidamide (compound 1)

To 50 mL three-necked flask, 7.0 g (0.04 mol) 2-isobutylidene-N′-nitrohydrozinecarboximidamides and 30 mL anhydrous DMF were sequentially added. A drying tube was installed, magnetically stirring was run, and ice water bath was used to lower the temperature. The temperature was lowered to below 10° C., the solution of 70% by mass of sodium hydride in DMF was added in three times (wherein, the mass of added sodium hydride is 2.4 g (0.07 mol)), and the mixture was reacted for 1 hour. The solution of 11.5 g (0.08 mol) iodomethane and 30 mL anhydrous DMF was slowly added dropwise through a drop funnel. After the addition was complete, the ice water bath was removed, and the temperature was raised to room temperature naturally. After the mixture was reacted for 2 hours at room temperature, 150 mL water was added, and solids were precipitated. The resulting product was settled, filtered, washed with water, and dried to obtain 2.9 g colorless plate-like crystal (2-isobutylidene-N-methyl-N′-nitrohydrozine-carboximidamides) in 45% yield, which was recrystallized with ethanol-petroleum ether (1:2 by volume), and its melting point is 60-61° C.

Structural characteristic data are provided as follows:

¹HNMR (CDCl₃, δppm): 1.18-1.16 (q, 6H), 2.62-2.73 (m, 1H), 3.36 (d, 3H), 7.15-7.16 (t, 1H), 7.45 (s, 1H), 9.05 (s, 1H)

elemental analysis

| | C % | H % | N % |
|---|---|---|---|
| theoretical value: | 38.50 | 7.00 | 37.41 |
| measured value: | 38.53 | 6.89 | 37.35 |

### EXAMPLE 2

### The synthesis of 2-(2′-nitrobenzylidene)-N-propyl-N′-nitrohydrozinecarboximidamides (the compound ZNQ-103 in formula I, wherein R1 is propyl, R2 is hydrogen, and R3 is o-nitrophenyl)

### (1) The synthesis of 2-(2′-nitrobenzylidene)-N′-nitrohydrozinecarboximidamides

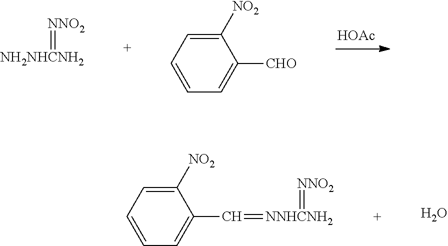

To 250 mL three-necked flask, 2.0 g (0.017 mol) N′-nitrohydrozinecarboximidamide, 100 mL anhydrous ethanol and 0.2 mL glacial acetic acid were sequentially added. It was heated to 65° C. under magnetic stirring, and the solution of 3.02 g (0.020 mol) o-nitrobenzaldehyde (the compounds in formula III, wherein R2 is hydrogen, and R3 is o-nitrophenyl) and 10 mL anhydrous ethanol was slowly added dropwise through a drop funnel. After the addition was complete, the mixture was heated to reflux, and refluxed for 3 hours. The temperature was lowered, the solvent was removed under the reduced pressure, and solids were precipitated. The resulting crude product was recrystallized with chloroform to obtain 3.21 g orange powder (2-(2′-nitrobenzylidene)-N′-nitrohydrozinecarboximidamide) in 75% yield, and the melting point is 225-226° C.

### (2) The synthesis of 2-(2′-nitrobenzylidene)-N-propyl-N′-nitrohydrozinecarboximidamides (ZNQ-103)

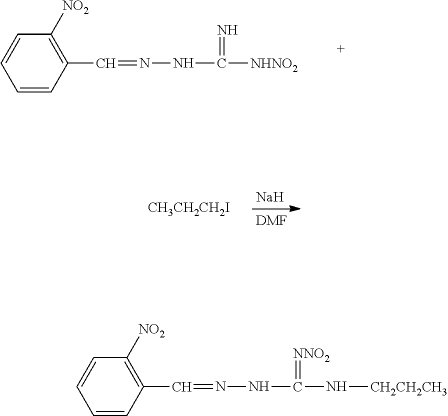

To 250 mL three-necked flask, 2.0 g (0.008 mol) 2-(2′-nitrobenzylidene)-N′-nitrohydrozinecarboximidamides and 50 mL anhydrous DMF were sequentially added. A drying tube was installed, magnetical stirring was run, and ice water bath was used to lower the temperature. The temperature was lowered to below 10° C., the solution of 70% by mass of sodium hydride in DMF was added in three times (wherein, the mass of added sodium hydride is 0.48 g (0.014 mol)), and the mixture was reacted for 1 hour. The solution of 2.72 g (0.016 mol) iodopropane and 10 mL anhydrous DMF was slowly added dropwise through a drop funnel. After the addition was complete, the ice water bath was removed, and the temperature was raised to room temperature naturally. After the mixture was reacted for 2 hours at room temperature, 150 mL water was added, and solids were precipitated. The resulting product was settled, filtered, washed with water, and dried to obtain 1.34 g yellow powder (2-(2′-nitrobenzylidene)-N-propyl-N′-nitrohydrozinecarboximidamides) in 57% yield, which was recrystallized with ethyl acetate with 158° C. melting point.

Structural characteristic data are provided as follows:

¹H NMR (DMSO-d₆, δppm): 3.89 (s, 3H, —CH₃), 4.79-4.82 (m, 2H, —CH₂—), 5.12-5.29 (m, 2H, —NCH₂), 6.94-7.04 (m, 2H, —ArH), 7.39-7.45 (m, 1H, —ArH), 7.80-7.83 (m, 1H, —ArH), 8.29 (s, 1H, —CH═N—), 9.16 (s, 2H, —NH-x2).

elemental analysis

| | C % | H % | N % |
|---|---|---|---|
| theoretical value: | 44.90 | 4.80 | 28.56 |
| measured value: | 44.90 | 4.73 | 28.51 |

### EXAMPLE 3

### Test on the Insecticidal Activity of Compounds of the Present Invention

Aphis, which belongs to Homoptera and has a piercing-sucking mouthpart, is a common pest for agricultural plant. The test subjects are myzus persicae, hyalopterus pruni, and aphis gossypii, and the test is performed by the way of immersing. Myzus persicae were derived from cabbage fields in Hai Dian district, Beijing, hyalopterus pruni were derived from peach tree in Dian district, Beijing, and aphis gossypii were derived from hibiscus trees in Hai Dian district, Beijing. Each test was carried out using 3 day-old nymphae.

Operational procedure: 20 mg compounds provided by the present invention (calculating based on 100% content) was exactly weighed and formulated into 0.5% by mass of stock solution with 4 mL acetone. Then, the stock solution was formulated into a series of liquid medicine to be determined using aqueous solution containing 0.1% by mass of Triton X-100. The leaves with aphids were chosen, and 3-day-old nymphae were left. After the leaves with aphids were immersed into the liquid medicine for 5 seconds and air-dried, the amount of aphids was recorded, and aphids were put into the culture dishes with moistened filter paper, then the culture dishes were capped and put into light incubator at (25±1)° C. Each of medicament treated 30 or more aphids, while the blank controls being set up. After 5-48 hours, the results were examined.

The criteria for death judgment is: slightly touching the bodies of the pest, the one which can not normally creep is considered as dead individual.

Corrected mortality (%)=(mortality of samples-mortality of blank controls)/(1-mortality of blank controls)×100%.

Aleyrodids belong to homoptera pests and has a piercing-sucking mouthpart. Bemisia tabaci Gennadius was tested by the way of spraying.

Operational procedure: firstly, the samples were dissolved with dimethyl sulfoxide, then were formulated into a solution at concentration of 500 mg/L using distilled water solution containing 0.01% by mass of Triton, and clean water was used as a blank control. The leaf of cotton was drilled using a punch to generate a leaf discs with a diameter of 18 mm, and the drilled leaf disc was immersed into liquid medicine for 5 seconds, then air-dried at room temperature. Following about 2 hours, the leaf disc was placed back upwards on the bottom of digitiform tube plated with agar (the concentration is 1.4%). The treated digitiform tube was inversely placed on top of bemisia tabaci gennadius (eclosion was carried out for 24 hours), and then leaf was flicked, bemisia tabaci gennadius can automatically fly into the tube. Each tube can collect 25 bemisia tabaci gennadius. The tube orifice was packed (wrapped) with gauze, and then the tube was inversely placed in an insectarium for normal breeding. The breeding conditions are: L/D (photoperiod) is 14:10, T (temperature) is 26±2° C., RH (relative humidity) is 75±5%. After 1 hour, the condition of the test-insect was examined. If a test-insect was dead, then the test insect was not counted. After 48 hours, the results were collected.

Helicoverpa armigera belongs to noctuidae of Lepidoptera and has a chewing mouthpart. The compound sample was weighted using a balance with 0.0001 accuracy, and was formulated into a stock solution with Dimethylformamide (DMF). Then, the stock solution was formulated into a liquid medicine to be determined using aqueous solution containing 0.1% by mass of Triton X-100.

Operational procedure: the cotton leaves were rinsed, and clean amaranth leaves were drilled using a punch to generate a leaf disc of 2 cm diameter, then the leaf disc was immersed into the liquid medicine for 10 seconds. After air-dried, the leaf disc was placed into ten-well test box. 2-day-old larvae of asparagus caterpillar were inoculated, one larva for each well, and the plastic wrap was covered. After being capped, the test box was placed into a light incubator at the temperature of (27±1)° C. After 48 hours, the results were examined. The individuals which body response abnormally or irresponsively to slightly touching with a hand setting (dial needle) were deemed as dead.

Asparagus caterpillar belongs to noctuidae of Lepidoptera and has a chewing mouthpart. The liquid medicine was formulated using medicament: the compound sample was weighted using a balance with 0.0001 accuracy, and formulated into stock solution with dimethylformamide (DMF). Then, the stock solution was formulated into the liquid medicine to be determined using aqueous solution containing 0.1% by mass of Triton X-100.

Operational procedure: the amaranth leaves were rinsed, and clean amaranth leaves were drilled using a punch to generate a leaf disc of 2 cm diameter, then the leaf disc was immersed into the liquid medicine for 10 seconds. After air-dried, the leaf disc was placed into ten-well test box. 2-day-old larvae of asparagus caterpillar were inoculated; one larva for each well, and the plastic wrap was covered. After being capped, the test box was placed into a light incubator at the temperature of (27±1)° C. After 48 hours, the results were examined. The individuals which body response abnormally or irresponsively to slightly touching with a hand setting (dial needle) were deemed as dead.

The results were shown in Table 1-4 below.

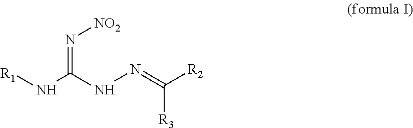

TABLE 1
The results of mortality of compounds of formula I to various pests.

| | | | | mortality(%) | | | |
|---|---|---|---|---|---|---|---|
| | | | | | bemisia | | |
| | | | | | tabaci | Helicoverpa | asparagus |
| | | | | myzus | gennadius | armigera | caterpillar |
| | | | | persicae | (500 μg/ | (500 μg/ | (500 μg/ |
| No. | R₁ | R₂ | R₃ | (600 μg/mL) | mL) | mL) | mL) |
| 1 | CH₃ | H | CH(CH₃)₂ | 4.7 | | 40 | 40 |
| 2 | CH₂CH₃ | H | CH(CH₃)₂ | 53.6 | | 60 | 50 |
| 3 | CH₂CH₂CH₃ | H | CH(CH₃)₂ | 14.4 | | 30 | 20 |
| 4 | (CH₂)₃CH₃ | H | CH(CH₃)₂ | 31.6 | | 10 | 20 |
| 5 | CH₂CH═CH₂ | H | CH(CH₃)₂ | 27.8 | | 90 | 30 |
| 6 | CH₂C≡CH | H | CH(CH₃)₂ | 37.9 | | 100 | 60 |
| 7 | CH₂Ph | H | CH(CH₃)₂ | 18.4 | | 40 | 50 |
| 8 | 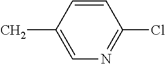 | H | CH(CH₃)₂ | 87.0 | | 60 | 40 |
| 9 | CH₂CH₃ | H | CH₂CH₃ | 54.2 | | 60 | 20 |
| 10 | CH₂CH₂CH₃ | H | CH₂CH₃ | 78.3 | | 80 | 20 |
| 11 | (CH₂)₃CH₃ | H | CH₂CH₃ | 1.5 | | 50 | 50 |
| 12 | CH₂CH═CH₂ | H | CH₂CH₃ | / | | 30 | 40 |
| 13 | CH₂C≡CH | H | CH₂CH₃ | 5.0 | | 50 | 0 |
| 14 | CH₂Ph | H | CH₂CH₃ | 18.8 | | 40 | 30 |
| 15 | 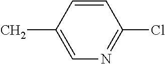 | H | CH₂CH₃ | / | | 10 | 20 |
| 16 | CH₃ | H | 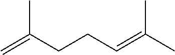 | 31.4 | | 30 | 70 |
| 17 | CH₂CH₃ | H | 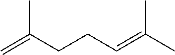 | 19.8 | | 40 | 40 |
| 18 | CH₂CH₂CH₃ | H | 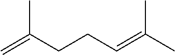 | / | | 50 | 60 |
| 19 | (CH₂)₃CH₃ | H | 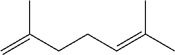 | 67.5 | | 40 | 60 |
| 20 | CH₂CH=CH₂ | H | 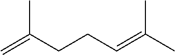 | 5.2 | | 0 | 50 |
| 21 | CH₂C≡CH | H | 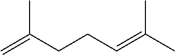 | 12.8 | | 20 | 30 |
| 22 | CH₂Ph | H | 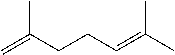 | 7.1 | | 20 | 20 |
| 23 | 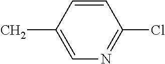 | H | 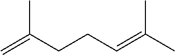 | 19.8 | | 30 | 40 |
| 24 | CH₂CH₃ | H | CH₂CH₂CH₃ | 4.7 | | 40 | 0 |
| 25 | CH₂CH₂CH₃ | H | CH₂CH₂CH₃ | / | | 50 | 10 |
| 26 | (CH₂)₃CH₃ | H | CH₂CH₂CH₃ | 65.3 | | 20 | 60 |
| 27 | CH₂CH═CH₂ | H | CH₂CH₂CH₃ | 65.1 | | 30 | 40 |
| 28 | CH₂C≡CH | H | CH₂CH₂CH₃ | 11.4 | | 60 | 70 |
| 29 | CH₂Ph | H | CH₂CH₂CH₃ | / | | 60 | 50 |
| 30 | 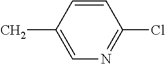 | H | CH₂CH₂CH₃ | 80.7 | | 40 | 40 |
| 31 | CH₃ | H | CH═CHCH₃ | 42.6 | | 60 | 10 |
| 32 | CH₂CH₃ | H | CH═CHCH₃ | 48.0 | | 0 | 0 |
| 33 | CH₂CH₂CH₃ | H | CH═CHCH₃ | 19.5 | | 70 | 70 |
| 34 | (CH₂)₃CH₃ | H | CH═CHCH₃ | / | | 40 | 60 |
| 35 | CH₂CH═CH₂ | H | CH═CHCH₃ | 56.7 | | 20 | 60 |
| 36 | CH₂C≡CH | H | CH═CHCH₃ | 35.0 | | 30 | 50 |
| 37 | CH₂Ph | H | CH═CHCH₃ | / | | 70 | 20 |
| 38 | 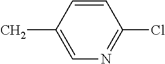 | H | CH═CHCH₃ | 1.2 | | 30 | 0 |
| 39 | CH₂CH₃ | H | CH═CH₂ | / | | 20 | 40 |
| 40 | CH₂CH₂CH₃ | H | CH═CH₂ | 8.3 | | 30 | 20 |
| 41 | (CH₂)₃CH₃ | H | CH═CH₂ | 74.7 | | 20 | 10 |
| 42 | CH₂CH═CH₂ | H | CH═CH₂ | 37.1 | | 20 | 0 |
| 43 | CH₂C≡CH | H | CH═CH₂ | / | | 40 | 20 |
| 44 | CH₂Ph | H | CH═CH₂ | 45.8 | | 30 | 0 |
| 45 | CH₂CH₂CH₃ | H | (CH₂)₅CH₃ | 16.9 | | 20 | 20 |
| 46 | (CH2)3CH3 | H | (CH₂)₅CH₃ | / | | 20 | 30 |
| 47 | CH₂CH═CH₂ | H | (CH₂)₅CH₃ | 23.7 | | 0 | 0 |
| 48 | CH₂C≡CH | H | (CH₂)₅CH₃ | 13.0 | | 30 | 0 |
| 49 | CH₂Ph | H | (CH₂)₅CH₃ | 28.6 | | 50 | 0 |
| 50 | 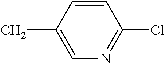 | H | (CH₂)₅CH₃ | 100 | | 20 | 0 |
| 51 | CH₂CH₂CH₃ | H | (CH₂)₃CH₃ | 13.3 | | 30 | 10 |
| 52 | (CH₂)₃CH₃ | H | (CH₂)₃CH₃ | 81.0 | | 20 | 60 |
| 53 | CH₂CH═CH₂ | H | (CH₂)₃CH₃ | 81.0 | | 40 | 80 |
| 54 | CH₂C≡CH | H | (CH₂)₃CH₃ | 47.6 | | 40 | 10 |
| 55 | CH₂Ph | H | (CH₂)₃CH₃ | 49.2 | | 10 | 30 |
| 56 | 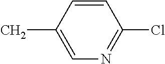 | H | (CH₂)₃CH₃ | 100 | | 70 | 20 |
| 57 | CH₃ | H | 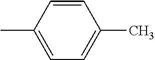 | 4.3 | | 20 | 20 |
| 58 | CH₂CH₃ | H | 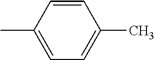 | 86.0 | | 30 | 30 |
| 59 | CH₂CH₂CH₃ | H | 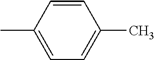 | / | | 50 | 0 |
| 60 | (CH₂)₃CH₃ | H | 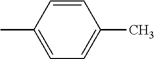 | 93.2 | | 0 | 0 |
| 61 | CH₂CH═CH₂ | H | 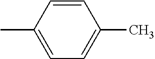 | 82.4 | | 80 | 80 |
| 62 | CH₂C≡CH | H | 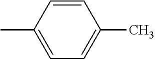 | 8.1 | | 60 | 60 |
| 63 | CH₂CH₃ | H | 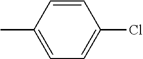 | / | | 10 | 100 |
| 64 | CH₂CH₂CH₃ | H | 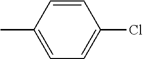 | 35.6 | | 50 | 50 |
| 65 | (CH₂)₃CH₃ | H | 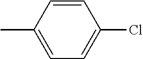 | 1.5 | | 0 | 60 |
| 66 | CH₂Ph | H | 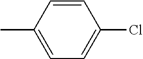 | 33.2 | | 10 | 70 |
| 67 | CH₂CH═CH₂ | H | 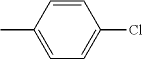 | / | | 10 | 50 |
| 68 | CH₂≡CH | H | 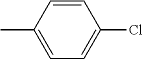 | 37.4 | | 0 | 70 |
| 69 | CH₂CH₃ | H | 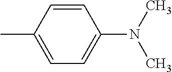 | 90.6 | | 30 | 80 |
| 70 | CH₂CH₂CH₃ | H | 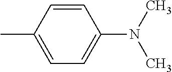 | / | | 40 | 80 |
| 71 | CH₂Ph | H | 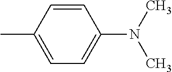 | / | | 30 | 90 |
| 72 | CH₂CH═CH₂ | H | 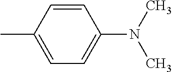 | 23.6 | | 50 | 50 |
| 73 | CH₂CH₂CH₃ | H | 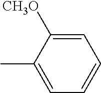 | 20.2 | | 50 | 70 |
| 74 | (CH₂)₃CH₃ | H | 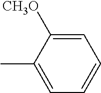 | 40.4 | | 0 | 30 |
| 75 | CH₂Ph | H | 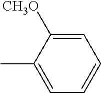 | 92.8 | | 30 | 90 |
| 76 | CH₂CH═CH₂ | H | 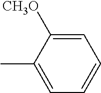 | 3.4 | | 40 | 80 |
| 77 | CH₂C≡CH | H | 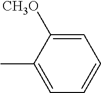 | 4.2 | | 30 | 50 |
| 78 | CH₃ | H | 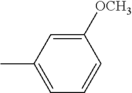 | 48.3 | | 80 | 60 |
| 79 | CH₂CH₃ | H | 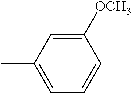 | 17.3 | | 0 | 60 |
| 80 | CH₂CH₂CH₃ | H | 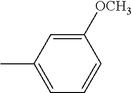 | / | | 50 | 70 |
| 81 | (CH₂)₃CH₃ | H | 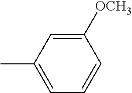 | 65.2 | | 40 | 60 |
| 82 | CH₂Ph | H | 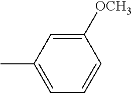 | / | | 60 | 50 |
| 83 | CH₂CH═CH₂ | H | 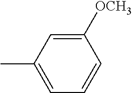 | / | | 20 | 50 |
| 84 | CH₂C≡CH | H | 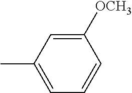 | 35.0 | | 10 | 70 |
| 85 | CH₂CH₃ | H | 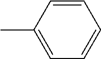 | 51.5 | | 20 | 40 |
| 86 | CH₂CH₂CH₃ | H | 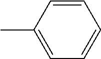 | 14.8 | | 30 | 80 |
| 87 | (CH₂)₃CH₃ | H | 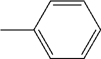 | 96.4 | | 10 | 50 |
| 88 | CH₂Ph | H | 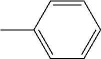 | 20.9 | | 10 | 30 |
| 89 | CH₂CH═CH₂ | H | 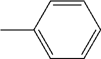 | 30.6 | | 10 | 80 |
| 90 | CH₂CH₂CH₃ | H | 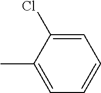 | 41.2 | | 10 | 50 |
| 91 | (CH₂)₃CH₃ | H | 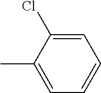 | 1.2 | | 0 | 80 |
| 92 | CH₂Ph | H | 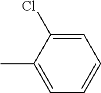 | 45.8 | | 50 | 70 |
| 93 | CH₂CH═CH₂ | H | 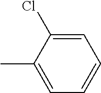 | 96.0 | | 40 | 70 |
| 94 | CH₂C≡CH | H | 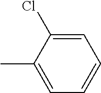 | 31.2 | | 50 | 60 |
| 95 | CH₃ | H | 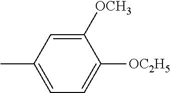 | 58.0 | | 40 | 80 |
| 96 | CH₂CH₂CH₃ | H | 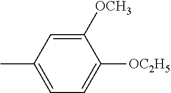 | 73.4 | | 20 | 50 |
| 97 | (CH₂)₃CH₃ | H | 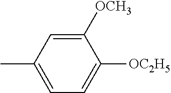 | 35.8 | | 40 | 70 |
| 98 | CH₂Ph | H | 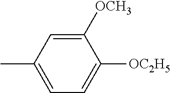 | 13.3 | | 40 | 10 |
| 99 | CH₂CH═CH₂ | H | 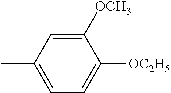 | 45.8 | | 50 | 30 |
| 100 | CH₂CH₂CH₃ | H | 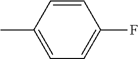 | 20.3 | | 20 | 90 |
| 101 | CH₂CH═CH₂ | H | 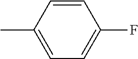 | 17.7 | | 40 | 30 |
| 102 | CH₂C≡CH | H | 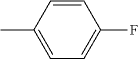 | 31.6 | | 50 | 50 |
| 103 | CH₂CH₂CH₃ | H | 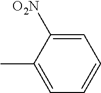 | 31.5 | | 20 | 60 |
| 104 | CH₂CH═CH₂ | H | 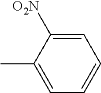 | / | | 40 | 50 |
| 105 | CH₂CH₂CH₃ | H | 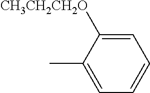 | 2.2 | | 50 | 50 |
| 106 | (CH₂)₃CH₃ | H | 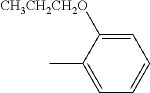 | 23.8 | | 30 | 30 |
| 107 | 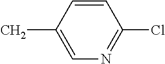 | H | 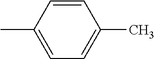 | 3.7 | | 10 | 20 |
| 108 | 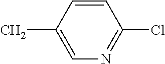 | H | 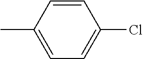 | / | | 50 | 50 |
| 109 | 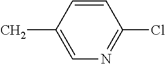 | H | 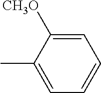 | / | | 40 | 90 |
| 110 | 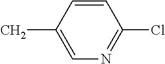 | H | 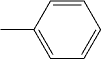 | 92.8 | 6.67 | 0 | 30 |
| 111 | 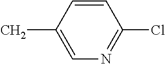 | H | 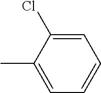 | 22.0 | 13.89 | 0 | 40 |
| 112 | 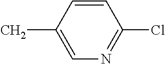 | H | 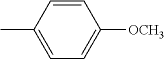 | 54.4 | 3.77 | 10 | 90 |
| 113 | 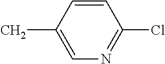 | H | 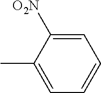 | 100 | 10.00 | 50 | 70 |
| 114 | 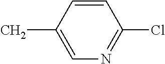 | H | 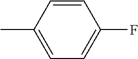 | 72.3 | 2.04 | 40 | 70 |
| 115 | 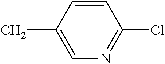 | H | 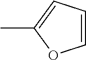 | 86.0 | 9.30 | 10 | 80 |
| 116 | CH₃ | H | 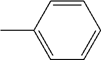 | | 10.29 | | |
| 117 | CH₃ | H | 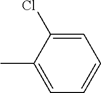 | | 17.78 | | |
| 118 | CH₃ | H | 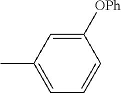 | | 14.81 | | |
| 119 | CH₃ | H | 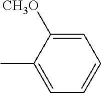 | | 72.00 | | |
| 120 | CH₃ | H | 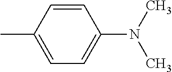 | | 82.76 | | |
| 121 | CH₃ | H | 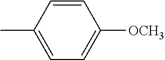 | | 16.95 | | |
| 122 | CH₃ | H | 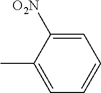 | | 21.82 | | |
| 123 | CH₃ | H | 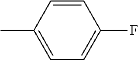 | | 4.69 | | |
| 124 | CH₃ | H | 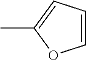 | | 88.68 | | |
| 125 | CH₂CH₃ | H | 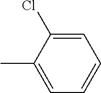 | | 53.06 | | |
| 126 | CH₂CH₃ | H | 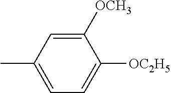 | | 6.67 | | |
| 127 | CH₂CH₃ | H | 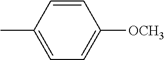 | | 24.56 | | |
| 128 | CH₂CH₃ | H | 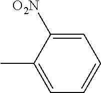 | | 19.05 | | |
| 129 | CH₂CH₃ | H | 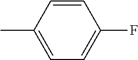 | | 8.62 | | |
| 130 | CH₂CH₂CH₃ | H | 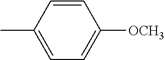 | | 18.46 | | |
| 131 | CH₂Ph | H | 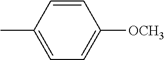 | | 1.89 | | |
| 132 | CH₂Ph | H | 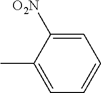 | | 9.68 | | |
| 133 | CH₂Ph | H | 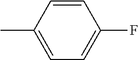 | | 3.77 | | |
| 134 | 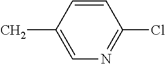 | H | 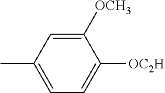 | | 1.82 | | |

TABLE 2
The insecticidal activity of the compounds of formula I to myzus persicae (24 h).

| Compound | Item | 400 μg/mL | 200 μg/mL | 100 μg/mL | 50 μg/mL | 25 μg/mL |
|---|---|---|---|---|---|---|
| 50 | numbers of | 96/96 | 103/107 | 87/87 | 91/92 | 78/78 |
| | death/total | | | | | |
| | numbers | | | | | |
| | mortality(%) | 100 | 96.3 | 100 | 98.9 | 100 |
| 56 | numbers of | 77/77 | 81/81 | 149/149 | 96/99 | 104/104 |
| | death/total | | | | | |
| | numbers | | | | | |
| | mortality(%) | 100 | 100 | 100 | 97.0 | 100 |
| 87 | numbers of | 74/74 | 77/77 | 77/78 | 79/81 | 56/84 |
| | death/total | | | | | |
| | numbers | | | | | |
| | mortality(%) | 100 | 100 | 98.7 | 97.5 | 66.7 |
| 93 | numbers of | 93/93 | 114/114 | 104/104 | 94/98 | 112/112 |
| | death/total | | | | | |
| | numbers | | | | | |
| | mortality(%) | 100 | 100 | 100 | 95.9 | 100 |
| 113 | numbers of | 84/88 | 92/92 | 85/85 | 66/68 | 48/80 |
| | death/total | | | | | |
| | numbers | | | | | |
| | mortality(%) | 95.5 | 100 | 100 | 97.1 | 60.0 |

| | | |
|---|---|---|
| CK | numbers of | 6/129 |
| | death/total | |
| | numbers | |
| | mortality(%) | 4.7 |

TABLE 3
The insecticidal activity of the compounds of formula I to hyalopterus pruni (24 h).

| Compound | Item | 400 μg/mL | 200 μg/mL | 100 μg/mL | 50 μg/mL | 25 μg/mL |
|---|---|---|---|---|---|---|
| 50 | numbers of | 102/102 | 110/110 | 101/101 | 98/102 | 104/108 |
| | death/total | | | | | |
| | numbers | | | | | |
| | mortality(%) | 100 | 100 | 100 | 96.1 | 96.3 |
| 56 | numbers of | 103/103 | 101/101 | 121/121 | 96/101 | 100/104 |
| | death/total | | | | | |
| | numbers | | | | | |
| | mortality(%) | 100 | 100 | 100 | 95.0 | 96.2 |
| 87 | numbers of | 99/99 | 107/107 | 110/114 | 94/101 | 90/114 |
| | death/total | | | | | |
| | numbers | | | | | |
| | mortality(%) | 100 | 100 | 96.5 | 93.1 | 78.9 |
| 93 | numbers of | 103/103 | 110/110 | 103/103 | 104/108 | 102/112 |
| | death/total | | | | | |
| | numbers | | | | | |
| | mortality(%) | 100 | 100 | 100 | 96.3 | 91.1 |
| 113 | numbers of | 114/114 | 102/102 | 105/107 | 106/110 | 88/118 |
| | death/total | | | | | |
| | numbers | | | | | |
| | mortality(%) | 100 | 100 | 98.1 | 96.4 | 74.6 |

| | | |
|---|---|---|
| CK | numbers of | 11/226 |
| | death/total | |
| | numbers | |
| | mortality(%) | 4.9 |

TABLE 4
The insecticidal activity of the compounds
of formula I to aphis gossypii (24 h).

| compound | item | 50 μg/mL | 12.5 μg/mL | 3.13 μg/mL |
|---|---|---|---|---|
| 50 | numbers of | 245/245 | 150/150 | 156/157 |
| | death/total | | | |
| | numbers | | | |
| | mortality(%) | 100 | 100 | 99.4 |
| 56 | numbers of | 166/178 | 136/164 | 121/157 |
| | death/total | | | |
| | numbers | | | |
| | mortality(%) | 93.3 | 82.9 | 77.1 |
| 87 | numbers of | 99/104 | 99/114 | 141/185 |
| | death/total | | | |
| | numbers | | | |
| | mortality(%) | 95.2 | 86.8 | 76.2 |
| 93 | numbers of | 138/138 | 124/124 | 178/186 |
| | death/total | | | |
| | numbers | | | |
| | mortality(%) | 100 | 100 | 95.7 |
| 113 | numbers of | 176/176 | 132/132 | 104/121 |
| | death/total | | | |
| | numbers | | | |
| | mortality(%) | 100 | 100 | 86.0 |

| | | |
|---|---|---|
| CK | numbers of | 1/85 |
| | death/total | |
| | numbers | |
| | mortality(%) | 1.2 |

### INDUSTRIAL APPLICATION

The present provides hydrocarbylidene nitrohydrozinecarboximidamides of structural general formula shown as formula I, and the use thereof as well as the method for making the same. These compounds were prepared by the inventors of the present invention through broadly investigation and rationally designing, as well as screening lots of compounds. Screened compounds have high insecticidal activity, and are prepared easily and conveniently. In addition, the present invention provides a preferred processing route, which has high safety and low cost, thus making practical value of these compounds be greatly improved.

The experiments of insecticidal activity show that the hydrocarbylidene nitrohydrozinecarboximidamides shown by formula (I) have high preventive efficiency against insect pests of plants, such as aphid, plant hopper, Helicoverpa armigera, asparagus caterpillar, and the like, so these compounds can be used as plant insecticides.

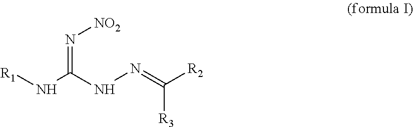

1. A compound shown by formula I or pharmaceutically acceptable salt thereof: 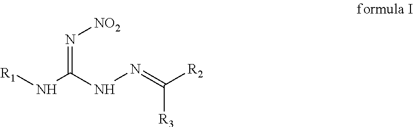
wherein
R1 is C1-C10 saturated and/or unsaturated aliphatic hydrocarbonyl, benzyl, substituted benzyl, halogenated picolyl, halogenated thiazolyl methyl, tetrahydrofuryl methyl or oxazolyl methyl;
R2 is hydrogen, C1-C5 saturated and/or unsaturated aliphatic hydrocarbonyl, phenyl, substituted phenyl, pyridyl or substituted pyridyl;
R3 is hydrogen, C1-C10 saturated and/or unsaturated aliphatic hydrocarbonyl, furyl, phenyl, substituted phenyl, benzyl or substituted benzyl.

2. The compound according to claim 1, characterized in that the R1 is C1-C10 unsaturated aliphatic hydrocarbonyl, halogenated picolyl, halogenated thiazolyl methyl or tetrahydrofuryl methyl, and preferably the R1 is allyl, propargyl or chloro-picolyl.

3. (canceled)

4. The compound according to claim 1, characterized in that the R2 is hydrogen or C1-C5 saturated and/or unsaturated aliphatic hydrocarbonyl, and preferably the R2 is hydrogen.

5. (canceled)

6. The compound according to any one of claims 1-5, characterized in that the R3 is substituted phenyl or C1-C10 saturated and/or unsaturated aliphatic hydrocarbonyl.

7. (canceled)

8. A method for making the compound of claim 1, the method comprises the following steps:
1) reacting nitroguanidine with hydrazine hydrate to form the compound shown by the structural general formula II; 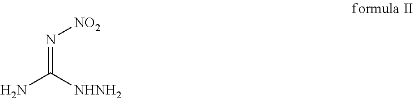
2) reacting the compound shown by the structural general formula II with the compound shown by the structural general formula III under acid catalysis, to form the compound shown by the structural general formula IV; 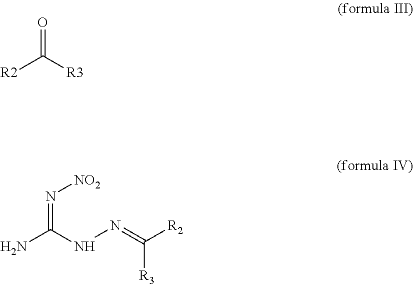
3) reacting the compound shown by the structural general formula IV with compound shown by the structural general formula V under alkalis catalysis, to form the compounds shown by the structural general formula I;
R1—X (formula V)
wherein, R2s of formula III and formula IV are hydrogen, C1-C5 saturated and/or unsaturated aliphatic hydrocarbonyl, phenyl, substituted phenyl, pyridyl or substituted pyridyl,
R3 is hydrogen, C1-C10 saturated and/or unsaturated aliphatic hydrocarbonyl, furyl, phenyl, substituted phenyl, benzyl or substituted benzyl,
R1 of formula V is C1-C10 saturated and/or unsaturated aliphatic hydrocarbonyl, benzyl, substituted benzyl, halogenated picolyl, halogenated thiazolyl methyl, tetrahydrofuryl methyl or oxazolyl methyl,
X of formula V is Cl, Br, I, 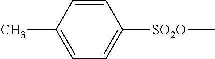
or F₃CSO₂O—

9. (canceled)

10. (canceled)

11. (canceled)

12. (canceled)

13. (canceled)

14. (canceled)

15. The method according to claim 8, characterized in that the reaction in step 1) is conducted in a solvent, and said solvent is water; and the reaction temperature of said reaction is 45-70° C.; the molar ratio of nitroguanidine to hydrazine hydrate in step 1) is (1:1)-(1:1.5).

16. The method according to claim 8, characterized in that the reaction in step 2) is conducted in a solvent, and said solvent is anhydrous ethanol or methanol; the reaction temperature of said reaction is 50-80° C.; the acid used in said reaction is acetic acid or p-toluenesulfonic acid; and the molar ratio of compound shown by the structural general formula II to compound shown by the structural general formula III in step 2) is (1:1)-(1:2).

17. The method according to claim 8, characterized in that the reaction in step 3) is conducted in a solvent, and said solvent is dimethylformamide or dimethylacetamide; the reaction temperature of said reaction is 0-50° C.; the alkali used in said reaction is sodium hydride, sodium ethoxide, sodium methoxide or sodium amide; and the molar ratio of the compound shown by the structural general formula IV to the compound shown by the structural general formula V in step 3) is (1:1.2)-(1:2.5).

18. (canceled)

19. (canceled)

20. (canceled)

21. A plant insecticidal drug or formulation whose active ingredient is the compound according to any one of claims 1, 2, 4, and 6 or pharmaceutically acceptable salt thereof and the active ingredient of the plant insecticidal drug or formulation is 0.01%-99.99% by mass.

22. (canceled)

23. The plant insecticidal drug or formulation according to claim 21, characterized in that the plant insecticidal drug or formulation is the drug or formulation that kills homoptera pests and/or lepidoptera pests including aphididae, aleyrodidae, delphacidae, psyllidae, jassidae, coccidae pests, noctuidae and/or plutellidae.

24. (canceled)

25. (canceled)

26. A method for preventing insect pests of plants by applying the plant insecticidal drug or formulation according to any one of claims 21 and 23 to plant leaves and/or plant fruits and/or plant seeds, and the places where the plant leaves and/or plant fruits and/or plant seeds are growing or are expected to be grown; the active ingredient of the plant insecticidal drug or formulation is administrated at the concentration of 1-600 mg/L.

27. The method according to claim 26, characterized in that the active ingredient of the plant insecticidal drug or formulation is administrated at the concentration of 3-50 mg/L.
